(19) United States
(12) Patent Application Publication
(10) Pub. No.: US 2002/0029956 A1
(43) Pub. Date: Mar. 14, 2002

(54) Method and apparatus for removing minute particles from a surface

(76) Inventor: Susan Davis Allen, Tallahassee, FL (US)

Correspondence Address: FLESHNER & KIM, LLP, P.O. Box 221200, Chantilly, VA, 20153-1200, US

(21) Appl. No.: 09/909,993

(22) Filed: Jul. 23, 2001

Related U.S. Application Data
(60) Provisional application No. 60/220,418, filed on Jul. 24, 2000.

(51) Int. Cl. C07C 6/00; B01J 19/08

(52) U.S. Cl. 204/157.15; 422/186

(57) ABSTRACT

A method and apparatus for removing one or more minute particle(s) from a surface of a sample using laser technology is provided. The laser energy wavelength, the pulse length and shape of the laser energy, the laser energy density, the pulse repetition rate of the laser energy, the laser beam size and/or shape, the irradiation geometry, the ambient conditions, the amount and disposition of the energy transfer medium, and/or the composition of the energy transfer medium are selected and controlled, based on application and environment considerations, to precisely control the energy deposition into the particle(s), sample, and/or the energy transfer medium combination.

### BACKGROUND OF THE INVENTION

1. Field of the Invention

The invention is directed to a method and apparatus for removing particles from a surface of a sample. More particularly, the invention is directed to a method and apparatus for removing minute particles from a surface of a sample using laser technology.

2. Background of the Related Art

Particle contamination of surfaces is a concern in many areas of technology. Two areas where such contamination can be a very significant problem are optics, particularly those with critical optical surfaces, and electronic device fabrication. The effect of contaminants on critical optical surfaces (coated or uncoated, dielectric or metal), for example in high power laser optics, can lead to increased optical absorption and a decreased laser damage threshold. Thus, as minute particles contaminate optical surfaces, they can serve as sinks for optical power incident on the optical surfaces and thus produce localized heating and possible damage. Large telescope mirrors, and space optics are other applications which require highly decontaminated critical optical surfaces.

In the electronics industry, particle contamination is an important factor in the manufacture of high density integrated circuits. Even in relatively conventional technology using micron or larger circuit patterns, submicron size particle contamination can be a problem. Today the technology is progressing into submicron pattern sizes, and particle contamination is even more of a problem. For device fabrication, particles serve as “killer defects” for only the device that is particle contaminated. The term “device” includes electronic devices, including masks/reticles, optical devices, medical devices, and other devices where particle removal could be advantageous. A particle contaminated mask/reticle prints every device with a defect. At the shorter wavelengths being developed for the next generation of lithography, materials for a protective pellicle for the mask are not available, making particle removal techniques an essential technology in the future. Contaminant particles larger than roughly 10% of the pattern size can create damage, such as pinholes, which interfere with fabrication processes (such as etching, deposition and the like), and defects of that size are a sufficiently significant proportion of the overall pattern size to result in rejected devices and reduced yield. As an example, it has been found that the minimum particle size which must be removed in order to achieve adequate yield in a one Megabit chip (which has a pattern size of one micron) is about 0.1 microns.

Filtration (of air and liquid), particle detection, and contaminant removal are known techniques used in contamination control technology in order to address the problems outlined above. For example, semiconductor fabrication is often conducted in clean rooms in which the air is highly filtered, the rooms are positively pressurized, and the personnel allowed into the room are decontaminated and specially garbed before entry is allowed. In spite of that, the manufactured devices can become contaminated, not only by contaminants carried in the air, but also by contaminants created by the processes used to fabricate the devices.

Removal techniques for contaminants should provide sufficient driving force for removal yet not destroy the substrate. Moreover, acceptable removal techniques should provide a minimum level of cleanliness in a reliable fashion. As the particle size decreases, the particle weight becomes less significant as compared to other adhesive forces binding the particle to the surface which it contaminates. Removal of such small particles can potentially damage the substrate.

In general, it has been found that submicron particles are the most difficult to remove. Many of the processes developed to clean integrated circuits, such as ultrasonic agitation, are not effective for micron and submicron particles and indeed, sometimes add contaminants to the substrate.

Laser assisted particle removal (LAPR) is a technique that has shown significant promise for removing minute, for example, both micrometer and nanometer scale, particles from critical surfaces, such as semiconductor wafers, high resolution photolithographic masks, high density magnetic recording media, large area high resolution optics and other critical surfaces. LAPR involves the rapid deposition of energy provided by lasers. Several different versions of LAPR exist depending on whether the laser energy is deposited in the particle, substrate or an energy transfer medium condensed under and around the particle.

The first Laser Assisted Particle Removal (LAPR) was probably observed in the early 1970s. Researchers who were studying the mechanisms of laser damage in materials for high power laser optics frequently observed and reported that a higher damage threshold was measured if one started at a low pulsed laser energy density and gradually increased the pulse energy until damage occurred (termed N/1, i.e., N shots on one site) as compared to the corresponding 1/1 experiments where each site was irradiated only once. The mechanism invoked for this damage threshold increase was surface cleaning during the initial low energy pulses. See, for example, S. D. Allen, J. O. Porteus, and W. N. Faith, Appl. Phys. Lett. 41, 416, 1982; S. D. Allen, J. O. Porteus, W. N. Faith, and j. B. Franck, Appl. Phys. Lett. 45, 997,1984; and j. O. Porteus, J. B. Franck, S. C. Seitel, and S. D. Allen, Optical Engineering 25, 1171, 1986, which are hereby incorporated by reference. During these N/1 experiments, particulate removal could be detected via a decrease in scattering of the alignment beam (usually He—Ne) and by bright “meteor” trails observed as the removed particle(s) traversed the He—Ne beam.

It was not until the late 1980s, however, that such LAPR began to be studied on its own merits, spurred in large part by the problem of particulates on semiconductor wafer surfaces creating defects in lithographic patterns. This problem remains, as discussed above, and the scale has shrunk significantly since the early work—from approximately 1 μm to 10 nm. Other critical surfaces which could benefit from an efficient LAPR system include: large area optics—both terrestrial and in space, masks for optical or x-ray lithography, electron or ion beam lithography, high density magnetic recording media, and high power laser optics.

Initial LAPR experiments concentrated on mechanisms whereby the expansion of the laser heated particle or substrate under the particle provided momentum to the particle normal to the surface, resulting in its removal. Imen et al. introduced in 1990 the idea of an energy transfer medium (ETM) that absorbs the laser energy either directly, see K. Imen, S. J. Lee, and S. D. Allen, Appl. Phys. Lett. 58, 203, 1991, which is hereby incorporated by reference, or by conduction from the substrate as shown by Zapka et al., see W. Zapka, W. Ziemlich, and A. C. Tam, Appl. Phys. Lett. 58, 2217,1991, which is hereby incorporated by reference. Many variations on these basic themes have subsequently been reported.

Laser assisted particle removal was described, for example, in U.S. Pat. No. 4,987,286 issued to Susan D. Allen on Jan. 22, 1991, which is hereby incorporated by reference. U.S. Pat. No. 4,987,286 discloses a method and apparatus for removing minute particles from a surface to which they are adhered using laser technology, and further teaches the use of an energy transfer medium to effect efficient laser assisted particle removal (LAPR). As shown in FIG. 5, a condensed liquid or solid energy transfer medium 23, such as water, is interposed under and around a contaminant particle 22 to be removed from a substrate 20 to which the particle is adhered. Thereafter, the medium 23 is irradiated using laser energy 25 at a wavelength which is strongly absorbed by the medium 23 causing explosive evaporation of the medium 23 with sufficient force to remove the particle 22 from the surface of the substrate.

Another particle removal technique has been to direct the laser energy into the substrate. The laser heated substrate then transfers energy into the energy transfer medium via conduction causing explosive evaporation sufficient to remove the particle from the surface of the substrate. Similarly, the laser energy can also be directed into the particle(s) to be removed.

Both direct absorption by the energy transfer medium, and substrate and/or particle(s) absorption with subsequent heating of the energy transfer medium can result in efficient LAPR and, as previously discussed, advances in technology have decreased the critical dimensions of various devices, such as, for example, magnetic hard drives, semiconductor devices, masks to make semiconductor devices, etc., and have also increased the surface quality requirements for devices such as large telescope mirrors, space optics, high power laser optics, etc. Therefore, the ability to remove particulate contamination in a noncontact clean fashion has become ever more important.

The above references are incorporated by reference herein where appropriate for appropriate teachings of additional or alternative details, features and/or technical background.

### SUMMARY OF THE INVENTION

An object of the invention is to solve at least the above problems and/or disadvantages and to provide at least the advantages described hereinafter.

The present invention relates to a method and apparatus for removing minute, for example, micrometer and nanometer scale, particles from a surface of a sample using laser technology. The laser wavelength, the pulse length and shape of the laser energy, the laser energy density, the pulse repetition rate of the laser energy, the laser beam size and/or shape, the irradiation geometry, the ambient conditions, the amount and disposition of the energy transfer medium, and/or the composition of the energy transfer medium are selected and controlled, based on application (i.e., substrate and pattern, particle composition size, and shape) and environment (i.e., external ambient composition, and pressure) considerations, to precisely control the energy deposition into the particle/sample/energy transfer medium system.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIG. 1 is a schematic diagram of laser assisted particle removal (LAPR) mechanisms;

FIG. 2 is a schematic diagram of energy flow in a mechanical LAPR system;

FIG. 3 is a table of experimental results obtained by Applicant and others using LAPR;

FIG. 4 is a graph showing cleaning efficiency versus laser fluence for several types of particles ranging from approximately 60 to 800 nm on Si;

FIG. 5 is a diagram schematically illustrating a contaminated surface with adhered particles illustrating the practice of laser assisted particle removal;

FIG. 6A is a diagram schematically illustrating a surface bearing a contaminant particle prior to the introduction of an energy transfer medium thereon;

FIG. 6B is a diagram schematically illustrating the introduction of the laser onto the particle contaminated surface after the energy transfer medium is disposed on the surface;

FIG. 6C is a diagram schematically illustrating the removal of the contaminant particle from the surface;

FIG. 7 is a diagram of a surface with a contaminant particle useful in understanding the present invention;

FIG. 8 is a schematic diagram of a system for performing the methods according to the present invention;

FIG. 9 is a schematic diagram of an alternative system for performing the methods according to the present invention;

FIG. 10 is a flow chart illustrating a method according to the present invention;

FIGS. 11-12 discloses a particle gun according to an embodiment of the invention; and

FIG. 13 discloses a particle gun according to another embodiment of the invention.

Similar reference numerals refer to similar parts throughout the several view of the drawings.

### DETAILED DESCRIPTION OF PREFERRED EMBODIMENTS

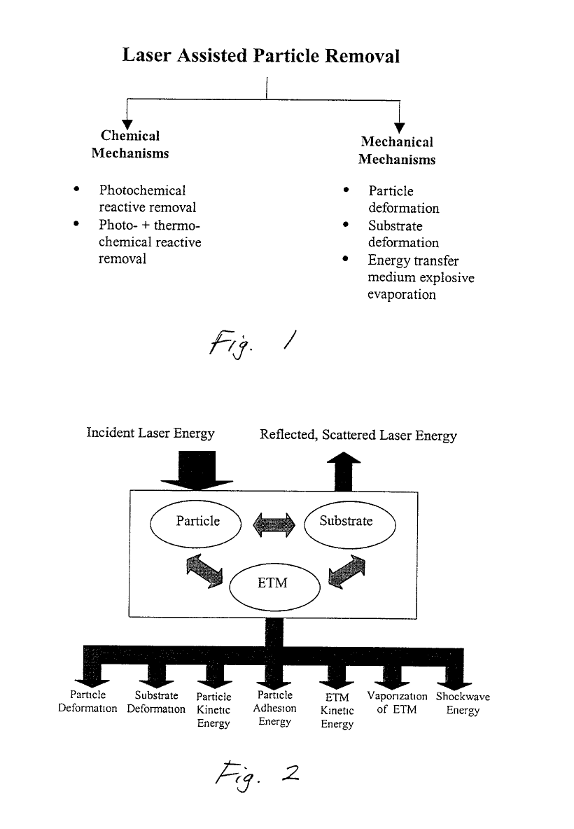

As shown in FIG. 1, LAPR can be roughly divided into chemical and mechanical removal mechanisms. In chemical removal, the laser energy can interact directly with the particle either photochemically or thermally. In the former case, the laser photochemically breaks down the molecules of the particle into vaporizable components. In the more common thermal case, the laser vaporizes the particle. The disadvantage in both of these approaches is the relatively high probability of reaction products further contaminating the substrate.

Alternatively, organic particles, in particular, can be removed from a substrate via reaction with an excited state species created by absorption of the laser into an appropriately chosen precursor. An example of such a system where the incident excimer laser is used as both a source of thermal energy and to create photochemically active scavenger species has been demonstrated by Oramir Semiconductor Equipment Ltd. See M. Genut, B. Livshits, Y. Uziel, O. Tehar-Zahav, E. Iskevitch, and I. Barzilay, Proc. SPIE 3274, 90, 1998; and D. Yogev, M. Engel, S. Zeid, I. Barzilay and B. Livshits, Proc. SPIE 3933, 77, 2000, which are hereby incorporated by reference.

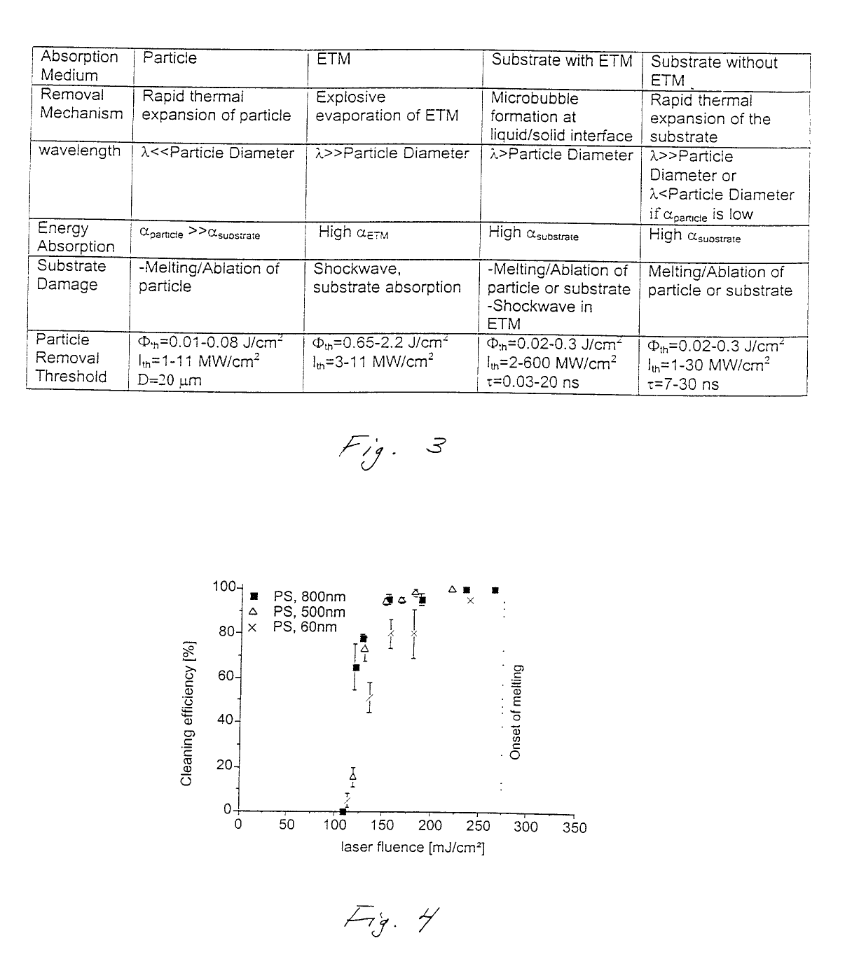

Most current LAPR methods rely on laser mechanical removal of the particles. A schematic of a composite mechanical LAPR system is shown in FIG. 2. The laser energy is absorbed into one or more of the three system components: particle, substrate, and energy transfer medium (ETM), if present. Each of these energy absorption sites will be discussed in turn. Experimental results obtained by the inventor and others are summarized in FIG. 3. See K. Imen, S. J. Lee, and S. D. Allen, Appl. Phys. Lett. 58, 203,1991; W. Zapka, W. Ziemlich, and A. C. Tam, Appl. Phys. Lett. 58, 2217,1991; J. D. Kelley, M. I. Stuff, F. E. Hovis, and G. J. Linford, Proc. SPIE 1415, 211, 1991; Y. F. Lu, W. D. Song, C. K. Tee, D. S-H. Chan, and T. S. Low, Jpn. J. Appl. Phys. 37,840, 1998; Y. F. Lu, W. D. Song, B. W. Ang, M. H. Hong, D. S -H. Chan, and T. S. Low, Appl. Phys. A 65, 9, 1997; A. C. Tam, W. P. Leung, W. Zapka, and W. Ziemlich, J. Appl. Phys. 71, 3515, 1992; A. Miller, S. j. Lee, S. D. Allen, Mater. Sci. Eng. B49, 85, 1997; M. Mosbacher, V. Dobler, J. Boneberg, and P. Leiderer, Appl. Phys. A 70, 669, 2000; S. j. Lee, K. Imen, and S. D. Allen, J. Appl. Phys. 74, 12, 1993; M. Mosbacher, H -J. Münzer, J. Zimmermann, J. Solis, J. Boneberg, and P. Leiderer, Appl. Phys. A 72, 41, 2001; S. J. Lee, K. Imen, and S. D. Allen, Appl. Phys. Lett. 61, 2314 (1992); D. R. Halfpenny and D. M. Kane, J. Appl. Phys. 86, 6641, 1999; J. B. Héroux, S. Boughaba, I. Ressejac, E. Sacher, and M. Meunier, J. Appl. Phys. 79, 2857, 1996; M. Mosbacher, N. Chaoui, J. Siegel, V. Dobler, J. Solis, J. Boneberg, C. N. Afonso, and P. Leiderer, Appl. Phys. A 69, 331, 1999; X. Wu, E. Sacher and M. Meunier, J. Appl. Phys. 87, 3618, 2000; G. Vereecke, E. Röhr, and M. M. Heyns, J. Appl. Phys. 85,3837, 1999; Y. F. Lu, W. D. Song, K. D. Ye, Y. P. Lee, D. S -H. Chan, and T. S. Low, Jpn. J. Appl. Phys. 36, L1304, 1997; Y. F. Lu, Y. W. Zheng, W. D. Song, Appl. Phys. A 68, 569, 1999; and K. Mann, B. Wolff-Rottke and F. Muller, Appl. Surf. Sci. 96-98, 463, 1996, which are hereby incorporated by reference.

A laser heated particle can be removed from a surface via a “hopping” mechanism generated by rapid thermal expansion. In order for this mechanism to occur, the laser wavelength (λ) must be much smaller than the particle diameter so that efficient particle heating can occur. Ideally, the absorption coefficient of the particle (α_{particle}) should be much greater than the absorption coefficient of the substrate (α_{substrate}) No energy transfer medium is present in this case. Removal of particles with diameters of a few micrometers has been observed for Tungsten (W) and up to several tens of micrometers for Aluminum (Al) and Copper (Cu) particles. Corresponding removal thresholds of approximately 0.65-2.1 J/cm² (30-90 MW/cm²) for W and approximately 10-80 mJ/cm² (approximately 1-11 MW/cm²) for Al and Cu have been observed. See J. D. Kelley, M. I. Stuff, F. E. Hovis, and G. J. Linford, Proc. SPIE 1415, 211, 1991; and Y. F. Lu, W. D. Song, C. K. Tee, D. S -H. Chan and T. S. Low, Jpn. J. Appl. Phys. 37, 840, 1998, which are hereby incorporated by reference. Directing laser energy into the particle(s)/substrate interface may also serve to break the bond holding the contaminant particle(s) to the substrate.

In an interesting experiment, Lu et al., reported that larger particles were more efficiently removed using backside irradiation through a transparent substrate. See Y. F. Lu, W. D. Song, B. W. Ang, M. H. Hong, D. S -H. Chan, and T. S. Low, Appl. Phys. A 65, 9, 1997, which is hereby incorporated by reference. This allows the laser heating and rapid expansion of the surface of the particle directly in contact with the substrate and should enhance the “hopping” mechanism.

A similar mechanism can be invoked to explain rapid laser heating of the substrate causing particle removal. This removal process is rather like having the particle on a rapidly rebounding trampoline. Measured and calculated surface expansion velocities and accelerations of rapidly heated laser surfaces exceed approximately 1 m/s, see A. C. Tam, W. P. Leung, W. Zapka and W. Ziemlich, J. Appl. Phys. 71, 3515, 1992; and V. Dobler, R. Oltra, J. P. Boquillon, M. Mosbacher, J. Boneberg, and P. Leiderer, Appl. Phys. A 69, 335, 1999, and approximately 10⁸ M/s², see M. She, Dongsik Kim, and C. P. Grigoropoulos, J. Appl. Phys. 86, 6519, 1999, which is hereby incorporated by reference, respectively. In this case, the laser wavelength (λ) should be greater than or equal to the particle diameter so that enough laser energy is diffracted around the particle to heat the surface under it. Alternatively, transparent particles would also allow the laser energy to be absorbed by the substrate. The substrate absorption coefficient (α_{substrate}) must be high so that the laser energy is concentrated in the surface, producing the maximum “bump” velocity/acceleration. Removal thresholds for a wide range of particle sizes and compositions range from approximately 20-300 mJ/cm² (approximately 1-30 MW/cm²) for pulse lengths of approximately 7-30 ns. Both visible and ultraviolet lasers have been used.

The addition of an ETM significantly lowers the removal threshold in most cases. As suggested by the acronym, the ETM serves to transfer the laser energy to kinetic energy of the particle perpendicular to the substrate surface. The ETM can be applied either as a uniform, thin film, or adsorbed under and around the adherent particle. The laser energy can be introduced directly into the ETM, or can be introduced into the substrate or particle, which then heats the ETM by conduction.

The ETM may be applied as a vapor that is allowed to condense on the substrate. Deposition of the ETM can be controlled thermodynamically or kinetically. In the former case, the temperature of the substrate is set and a controlled amount of the ETM is applied as a vapor that is allowed to condense on the substrate. After the condensation is complete, the system (particle(s)/substrate/ETM) is pulsed with laser energy. In the later case, the substrate is dosed with an overly sufficient amount of ETM. After a sufficient amount of ETM has evaporated from the surface of the substrate, the system is pulsed with laser energy. Depending on the dose and the delay time between the vapor dose and the laser pulse, the thickness of the film or amount under and around the particle can be controlled. For thin film deposition, optical monitoring of the thin film thickness using a visible cw laser can be used to control the thickness at which the laser pulse is triggered. See P. T. Leung, N. Do, L. Klees, W. P. Leung, F. Tong, L. Lam, W. Zapka, and A. C. Tam, J. Appl. Phys. 72, 2256, 1992, which are hereby incorporated by reference. For a similar control of the amount of ETM adsorbed under and around the particle, scattering of a visible laser from the particle/ETM system has been employed. See A. Miller, S. J. Lee, and S. D. Allen, Mater. Sci. Eng. B49, 85, 1997, which is hereby incorporated by reference.

Most work to date has used water or water/alcohol combinations as the ETM. Water was the first ETM used in LAPR, see K. Imen, S. J. Lee, and S. D. Allen, Appl. Phys. Lett. 58, 203, 1991, which is hereby incorporated by reference, and has the particular advantage of being able to store large amounts of energy before undergoing explosive evaporation. The kinetic limit of superheat for water is approximately 575 K, more than 200K above the boiling point. See C. T. Avedisian, J. Phys. Chem. Ref. Data 14, 695, 1985 and references therein, which are hereby incorporated by reference. Water is also advantageous in that it is a small molecule. An ETM formed of a smaller molecule can more readily diffuse into small spaces under and around the contaminant particle.

Thin uniform films have normally been used when the substrate is the primary absorber of the laser energy and the removal mechanism has been shown to be microbubble formation at the liquid/solid interface. See O. Yavas, P. Leiderer, H. K. Park, C. P. Grigoropoulos, C. C. Poon, W. P. Leung, N. Do, and A. C. Tam, Phys. Rev. Lett. 70, 1830, 1993; and A. C. Tam, H. K. Park, and C. P. Grigoropoulos, Appl. Surf. Sci. 127-129, 721, 1998, which are hereby incorporated by reference. Removal thresholds of approximately 20-300 mJ/cm² (approximately 2-600 MW/cm²) have been measured for visible and UV lasers with pulse lengths of approximately 30 ps-20 ns.

The thickness of the thin ETM film should be selected with consideration to the particle diameter, but it should not be so large that the laser pulse does not have enough energy to evaporate all of the ETM. The removal threshold should be a function of the substrate absorption with higher absorbing substrates having lower thresholds.

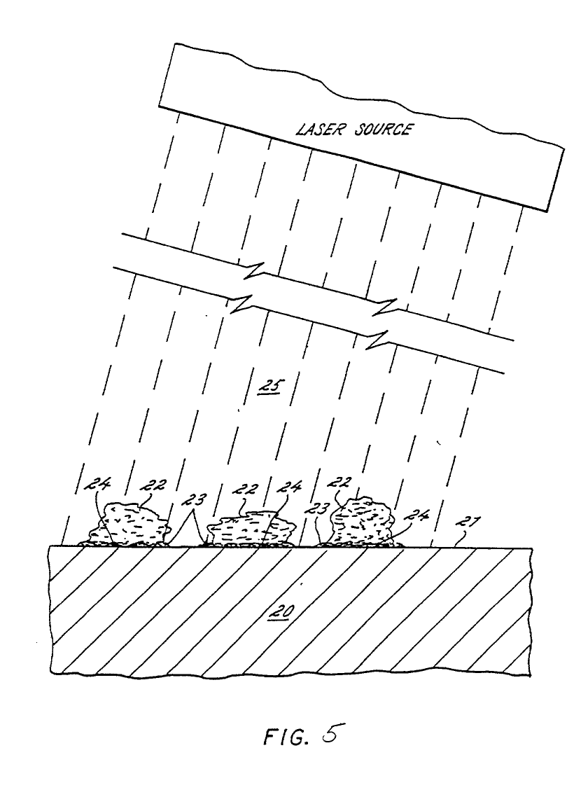

Most reported data to date have been for Si substrates, although different wavelength lasers have been used. For the same substrate, the threshold should not be a function of the particle size as long as the particle size is less than the wavelength of the LAPR laser so that efficient absorption in the substrate under the particle can occur. As shown in FIG. 4, M. Mosbacher et al. measured a “universal threshold” of approximately 110 mJ/cm² (14 MW/cm²) for an approximately 8 ns pulse of approximately 532 nm light for several types of particles ranging from approximately 60 to 800 nm on Si. See M. Mosbacher, V. Dobler, J. Boneberg, and P. Leiderer, Appl. Phys. A 70, 669, 2000, which is hereby incorporated by reference.

For laser wavelengths chosen such that most of the laser energy is absorbed in the ETM, the ETM can be applied either as a thin, uniform film, or as a droplet containing the particle. In this case the wavelength should also be greater than the particle diameter so that the laser energy is efficiently absorbed in the ETM under the particle. Explosive evaporation of the ETM acts as the removal mechanism. In fact, at laser energy densities significantly above the LAPR threshold, hemispherical shock waves are formed from the explosive evaporation. The threshold for shock wave formation has been measured as twice the LAPR threshold for CO₂ laser irradiation at approximately 9.6 and 10.6 μm wavelengths with a approximately 200 ns pulse length for several types of micrometer-scale particles on a silicon substrate. See S. J. Lee, K. Imen, and S. D. Allen, J. Appl. Phys. 74, 12, 1993, which is incorporated by reference. Thresholds of approximately 0.65-2.2 J/cm² (approximately 3-11 MW/cm²) have been measured for LAPR using a CO₂ laser with water as the ETM. See S. j. Lee, K. Imen, and S. D. Allen, J. Appl. Phys. 74, 12, 1993; and J. B. Héroux, S. Boughaba, I. Ressejac, E. Sacher, and M. Meunier, J. Appl. Phys. 79, 2857, 1996, which are hereby incorporated by reference.

It should be noted that a comparison of LAPR thresholds is not always straightforward. The definition of what constitutes the threshold depends on whether energy fluence or intensity is used, how the spot size and energy density or intensity are defined and how the cleaned area is defined. For example, while the maximum energy density threshold for CO₂ is significantly higher (approximately 2.2 J/cm²) than for an excimer laser (approximately 100-300 mJ/cm²), the pulse length is also longer (about approximately 200 ns for a CO₂ laser as compared to an excimer laser at about 20 ns). The intensity thresholds are, therefore, comparable. When the upper limit to the energy density is determined by substrate damage, the intensity is frequently the most appropriate measurement as short pulses damage more readily than long pulses in most substrate systems.

There is also the as-yet-unanswered question of the optimum pulse length. If the pulse is too long, rapid expansion of the particle or substrate or explosive evaporation of the ETM does not take place. For long pulses, too much of the laser energy diffuses away from the particle site. Pulses that are too short, on the other hand, may more readily cause substrate damage while not decreasing the removal threshold significantly. The optimum pulse length will undoubtedly depend on the specific laser/particle(s)/substrate/ETM involved.

Laser pulse energies or intensities are limited by damage to the substrate. In some cases, substrate damage may occur via decomposition of the particle or shockwave structural damage instead of or in addition to the more traditional melting and/or ablation of the substrate. Mosbacher et al. have reported damage via field enhancement around the particle, causing melting under the particles. See M. Mosbacher, H -J. Münzer, J. Zimmermann, J. Solis, J. Boneberg, and P. Leiderer, Appl. Phys. A 72, 41, 2001, which is hereby incorporated by reference.

It should be noted that the transmissivity or absorptivity of the substrate and particle(s) have a continuum of possibilities. That is, the substrate and/or particle(s) may be partially transmissive, the substrate may be completely transparent and the particle(s) partially absorptive and partially transparent, etc. This can be effected by the types of materials as well as application of films on the substrate, such as metallic thin film coatings or the like. Extreme Ultraviolet (EUV) optics will be largely reflective in nature as few or no transparent materials exist in the EUV. In addition, EUV lithography systems will operate in vacuum as gaseous ambients absorb EUV. LAPR can take place in vacuum and gaseous ambients.

The particle removal process can be used in both deep ultraviolet (DUV) lithography and EUV lithography. EUV optics are all-reflective. The laser parameters, such as power, pulse repetition rate, pulse form etc., are determined based on such things as the particle adhesion energy which in turn is a function of the surface properties of the substrate.

Removal thresholds and velocity distributions of the removed particles are a function of laser wavelength, pulse length, optical properties of the substrate and particles, and optical and thermal properties of the energy transfer medium. For the nanometer-scale thin film multilayers, e.g., Mo/Si, used for EUV reflectors, limiting the effective thermal input into the substrate will be an important factor.

The adhesion force between particles and surface is key to particle removal. The particles can be any type of particles, even, for example, bacteria or viruses where sterilization of a sample is desired. The particles on the surface adhere primarily due to van der Waals and capillary forces and, in some cases, due to chemical bonding effects. See R. G. Horn et al., Science, 256, 362, 1992, which is hereby incorporated by reference. In the case of physical interactions, the magnitude of this force depends on the particle size, Hamaker constant, surface roughness, and the relative humidity in the chamber. See J. N. Israelachvili, “Intermolecular Surfaces Forces”, Academic Press, London, 1992, which is hereby incorporated by reference.

An experimental set-up for LAPR threshold measurement has been outlined in previous papers. See S. K. Lee et al., “CO₂ Laser assisted particle removal threshold measurements,” Appl. Phys. Letter 61, 2314, 1992; S. K. Lee et al., “Shock wave analysis of laser particle removal,” J. Appl. Phys. 74, 12,1993; A. C. Tam et al., “Laser-cleaning techniques for removal of surface particles,” J. Appl. Phys. 71, 7, 1992; M. Mosbacher et al., “A comparison of ns and ps steam laser cleaning of Si surfaces,” Appl. Phys. A69, S 331-334, 1999; and M. Mosbacher et al., “Universal threshold for the steam laser cleaning of submicron spherical particles from silicon, Appl. Phys. A70, 669, 2000, which are hereby incorporated by reference. Cleaned area is measured after several ETM dose/laser pulse cycles using optical scattering of either white light (for relatively large particles) in dark field illumination geometry or using blue or ultraviolet light for smaller particles. Mosbacher et al. have shown that scattering is proportional to the number of even nanometer-scale particles. See M. Mosbacher et al., “Universal threshold for the steam laser cleaning of submicron spherical particles from silicon, Appl. Phys. A70, 669, 2000, which are hereby incorporated by reference. Particle removal thresholds will be a function of the following parameters:

Energy Transfer Medium Deposition—The amount and form of the ETM on the contaminated substrate at the irradiation site strongly affects the LAPR results. The dynamics of liquid accumulation on the substrate and around particles as a function of the dosing parameters are determined.

Energy Deposition—Optimum laser parameters must be determined for a particular application. Laser wavelength, λ, energy density, Φ, pulse length, τ, are the important variables. Choice of laser wavelength will determine the amount of absorption by the ETM and substrate. For example, for the Mo/Si multilayers currently used in the EUV, ETM absorption should be optimized and substrate absorption minimized. Appropriate pulsed laser sources include: CO₂(9-11 μm), Er:YAG (2.94 μm), and excimer (248, 193 and 157 nm). Other laser sources can be used as applicable. An optimum set of irradiation parameters result in delivery of: 1) a sufficient amount of laser energy in 2) a sufficiently short time to 3) the appropriate elements of the particle/substrate/ETM system. If the substrate absorbs significant amounts of laser energy, the LAPR threshold must be much lower than the damage threshold. If the pulse length is too long, explosive evaporation will not occur. If the pulse length is too short, the substrate may be damaged by the high peak laser powers.

Particle/Debris Removal Mechanisms—Ideally, the velocity of the removed particles should be sufficient to transport them far enough away from the cleaned substrate to prevent redeposition. Under atmospheric conditions, however the drag force exerted by air or other gaseous ambient on the small particles of interest is significant. Because EUV lithography tools will operate in vacuum, both various atmospheres and vacuum ambients must be considered as cleaning environments.

ETM Geometry—The actual ETM geometry and thermal conduction during the laser pulse is another relevant parameter in particle removal. Geometries can include: uniform layer of a particular thickness, droplet including the particle, and adsorption in the capillary space under the particle. The kinetic energy released when the superheated ETM undergoes explosive evaporation goes to overcome the particle adhesion energy and into the kinetic energy of the particles, gaseous ETM and substrate deformation and shock wave, if present. Assuming ideal behavior, the forces on the system exerted by the explosive evaporation and the resulting velocities are determined as a function of the extent of superheat and ETM geometry.

As previously discussed, a summary of the energy flow in LAPR is given schematically in FIG. 2 for laser mechanical removal mechanisms. The incident laser energy is absorbed into the particle(s), substrate and/or the energy transfer medium (if present) system with the weighting factors determined by the optical constants of the materials and the size of the particle(s) relative to the wavelength of the laser. Energy can be exchanged among the components, plus the surrounding gaseous or vacuum ambient, depending predominantly on the relative time scale of the processes. The outputs of the system can be: particle deformation, substrate deformation, particle kinetic energy, particle adhesion energy which must be overcome, enthalpy of vaporization of the ETM to form microbubbles or explosive evaporation, kinetic energy of the ETM and shockwave energy, if present. The challenge is to design the laser/ETM system to produce as much of the desired effects (for example, particle kinetic energy) while minimizing undesired effects (for example, shockwave formation).

The following parameters (which will be referred to as laser energy transfer parameters) that are variable within some limits must be optimized for industrial use:

1) Laser Wavelength, A—The laser wavelength should be chosen to target either the particle, the substrate, the ETM (if present) or some combination thereof.

2) Laser Beam Energy Density, φ—The energy density should be above the removal threshold but below the damage threshold.

3) Laser Pulse Length, τ—The laser pulse length should be short enough to create the desired effect but not any shorter in order to decrease the likelihood of substrate damage.

4) Beam Shape and Size—The ideal beam shape for an industrial process would be a uniform intensity beam as large as possible in order to clean as large an area as possible. Beam homogenizers are necessary for industrial application of these techniques. For measurements of thresholds, a Gaussian beam may be preferable as there is a simple relationship between the threshold and the maximum intensity in the beam. See S. J. Lee, K. Imen, and S. D. Allen, Appl. Phys. Lett. 61, 2314, 1992, which is hereby incorporated by reference.

5) Laser Pulse Rate—The laser pulse repetition rate should be selected to optimize the transfer of laser energy to the particle without causing damage to the substrate.

6) Energy Transfer Medium, ETM—Optical and thermophysical properties; as well as control and optimization of the amount deposited are the important parameters.

7) Irradiation Geometry—The irradiation geometry, that is, the component and direction in which the laser energy is directed should be selected to optimize the transfer of laser energy to the particle without causing damage to the substrate.

8) Ambient/Environment—The ambient/environment should be selected to control the composition and pressure.

An additional problem that must be solved before a successful industrial system is implemented is the prevention of redeposition of removed particles. The most fundamental requirement for redeposition prevention is that the velocity of the particle be greater than the escape velocity. As soon as the particle has been removed from the surface, it is subject to drag forces from the surrounding atmosphere and these retarding forces are more significant for smaller particles. Two solutions have been proposed and implemented to some degree—reduce the pressure of the surrounding ambient to increase the mean free path of the removed particle, or use a gas jet parallel to the surface to entrain the removed particle. It has also been suggested that the particles could be ionized and trapped electrostatically. Co-pending application Serial No.______[Attorney Docket No. FSU-0004] which is hereby incorporated by reference, discloses using thermophoresis to prevent redeposition of particles. Alternatively, a cold plate in a vacuum or low pressure ambient could be provided to draw removed particles (and any ETM) away from the surface and prevent them from redepositing. In any event, redeposition is another issue that much be addressed when utilizing LAPR.

In summary, LAPR is an attractive technique for the removal of small particles. Particles as small as approximately 60 nm have been successfully removed. See M. Mosbacher, V. Dobler, J. Boneberg, and P. Leiderer, Appl. Phys. A 70, 669, 2000, which are hereby incorporated by reference. Further, there is an increasing number of potential applications for the cleaning of critical surfaces. Exemplary embodiments of the methods and apparatus according to the invention will now be discussed.

As previously discussed, FIG. 5 shows, in cross-section, a portion of a substrate 20 bearing contaminant particles 22 which are adhered to a surface 21. The particles 22 are bound to the surface 21 by any of a number of forces. The particles are present usually as the result of a complex process which may include diffusion, sedimentation, inertia, and electrical or electrostatic attraction. When the particles are very small, for example, micrometer and nanometer scale, gravity is a minor source of adhesion, and other sources of greater significance are Van der Waals forces, electrostatic forces, capillary forces, and the like. Adhesion forces and the factors necessary for dislodging particles held by such forces will be considered in greater detail below. As the particles become smaller, the adhesion force per particle contact surface area increases rapidly, and removal of such particles becomes a rather significant problem.

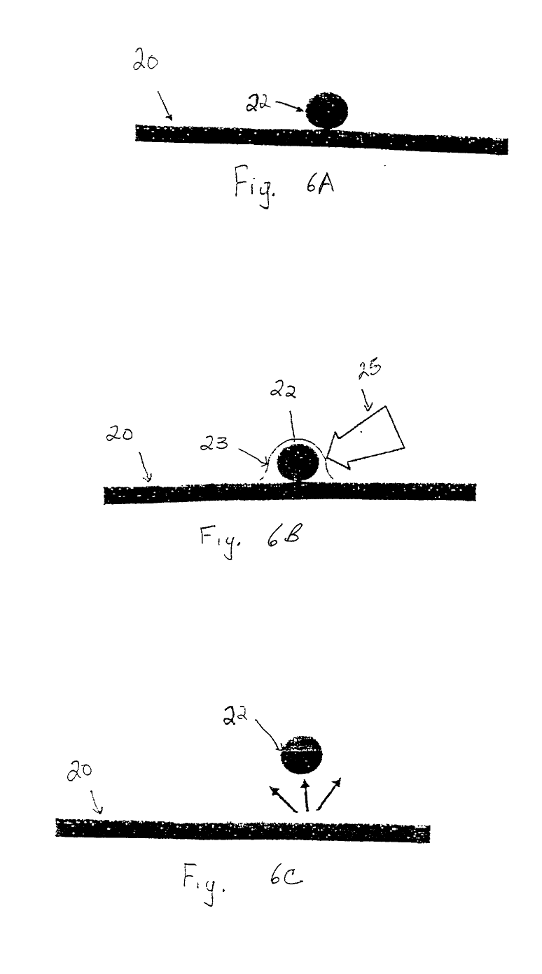

An energy transfer medium may be interposed between the surface 21 and the particles 22, such medium being illustrated in the drawing as layer 23, which occupies interstices 24 formed under and around the adhered particles 22. Other geometries, as discussed herein, may also be appropriate. FIGS. 6A-6B illustrates the introduction of the energy transfer medium onto a surface bearing a contaminant particle.

After preparing the surface for cleaning, energy is impinged upon the surface to be cleaned, such energy preferably being at a wavelength which is absorbed by the energy transfer medium, the substrate, particle(s), or a combination thereof. In FIG. 5, optical radiation from a laser beam 25 is directed at the surface 21 which carries the contaminant particles and interposed layer 24. A quantity of energy is absorbed in the energy transfer medium either directly or from the laser heated particles(s) or substrate, which is sufficient to cause explosive evaporation of the energy transfer medium. The quantity of material interposed under and around the particle is such that, when explosive evaporation occurs, the particle is driven from the surface by the force of the explosion, as shown in FIG. 6C. In effect, the laser energy incident on the surface is converted by the energy transfer medium to kinetic energy, and is transferred to the particle, driving it from the surface to which it had been adhered.

Means may be provided for collecting, or otherwise removing dislodged particles once freed from the surface so as to prevent the particles from redepositing on the surface. The explosive evaporation may occur with the substrate in a vacuum chamber, such that any dislodged particles are removed by means of vacuum creating equipment. As an alternative, a gas jet can be provided which impinges a stream of gas onto the surface to carry the dislodged particles away. In an application in space, vacuum is the natural ambient and the velocity imparted to the particles by explosive evaporation will be adequate to transfer the particles away from the surface. In any case, the requirement is simply for providing a velocity component to the particles which will carry the particles away from the surface to avoid recontamination.

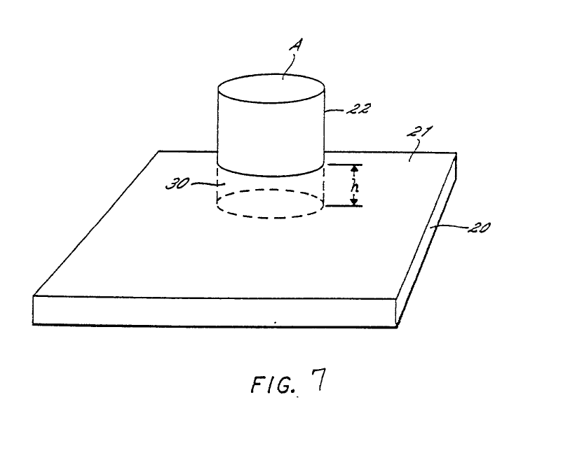

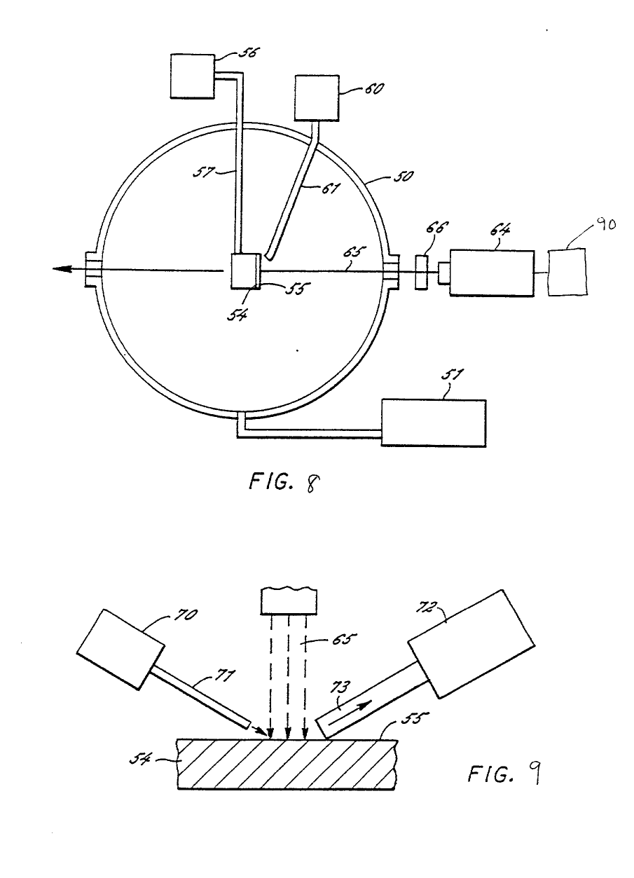

FIG. 7 illustrates the relationships for a system useful in determining such forces. Thus, there is shown in FIG. 7 a schematically illustrated substrate 20 having a surface 21 to which is bound a particle 22, shown for convenience as a cylindrical particle, although as will be appreciated the relationships similar to the simplified ones derived herein will be applicable to particles of any shape including those of irregular shape.

Disposed between the particle 22 and the surface 21 is a cylindrical volume 30 having a surface area equal to the surface area A of the particle and a height h. The volume 30 thus represents the energy transfer medium (of surface area A and height h), interposed between the particle 22 and surface 21.

A laser beam with fluence φ(J/cm²) is incident on the region that contains the particle and the interposed column. The dissipated energy density is φ/δ, where δ is the absorption depth of the laser beam in the material filling the column 30. For simplicity, it is assumed that the molecules of the energy transfer medium behave as an ideal gas and that the laser energy is instantly converted into thermal energy in the molecules. The equation governing the pressure (P) and volume (V) of n moles of an ideal gas is

PV=nRT.

The absorbed laser energy, transformed into kinetic energy of the molecules of the energy transfer medium, is related to temperature as:

E=m(vᵣₘₛ)²/2=(3/2)nRT

and to the laser fluence as

E=φV_{b}/δ,

where V_{b} is the volume of laser heated transfer medium. It will be appreciated from the foregoing that reflection and scattering is neglected. Combining the above expressions, the forces exerted by the molecules of the energy transfer medium on a particle of cross-sectional area A becomes

F=PA=2Aφ/3[Max.(h,γ)],

where [Max. (h,δ)] indicates that the expression should be evaluated for whichever h or δ is larger. As an example, the removal force for a one micron diameter particle, using an unfocused laser beam of intensity 0.1 J/cm², is 650 dynes (δ=0.8 microns and h<δ), which is roughly 3 orders of magnitude larger than the adhesion force binding a one micron particle to the surface. Furthermore, assuming that the substrate being cleaned is silicon, since the damage threshold of silicon is 55 J/cm², utilizing an intensity of only 0.1 J/cm² to assure particle removal allows a further margin of error of an additional 3 orders of magnitude before surface damage is encountered. As a further advantage, since the laser assisted particle removal forces described above are proportional to the cross-sectional area of the particle, i.e., r², use of this technique has a geometric advantage for smaller particles over conventional removal techniques which are proportional to the particle volume i.e., r³.

The foregoing has related the empirically determined force which binds a particle to the surface to the removal forces generated by explosive evaporation according to the present invention. It has been shown that a force can be generated when the invention is properly applied which is orders of magnitude greater than that which binds the particle to the surface, while still being orders of magnitude less than that capable of damaging the surface. The kinetic force which is brought to bear by the energy transfer medium as a result of absorbing energy and translating the absorbed energy to kinetic energy has been shown to be related to the laser fluence, the volume and shape of the liquid interface, and the absorption depth of the laser beam at the particular wavelength in the material of the energy transfer medium.

Certain refinements can be included in the foregoing model, although as will be appreciated the model is adequate for most purposes. With respect to one refinement, for small particles of interest, the laser energy is efficiently diffracted around the particle, allowing absorption of the bulk of energy by the energy transfer medium in the interstices. For larger particles, however, some of the medium in the interstices water can be shadowed by the particle, with the result being a decrease in coupling efficiency of the laser to the medium. The optical properties of the particle and substrate can also affect the energy absorption.

The major force which drives the particle removal mechanism, according to the invention, is the energy absorption by the energy transfer medium/substrate/particle(s) system, and transformation of that absorbed energy into kinetic energy. Conductive losses to the substrate are expected to be small and generally can be neglected. However, for certain irradiation protocols, conductive losses to the substrate may become a factor and should be considered.

Finally, in the foregoing, the explosive evaporation of the energy transfer is analyzed in the context of a spherical water droplet heated with a pulsed laser. In some cases, the reaction of the water interface to the pulsed laser may differ because of the capillary geometry, and consideration of that factor may prove necessary in a certain restricted number of cases.

Water provides a good energy transfer medium in that it is capable of significant superheating, thereby storing significant energy per volume. This stored energy is converted to kinetic energy on explosive evaporation and translated to the surface particle. Water is highly transparent at all of the visible and UV wavelengths longer than approximately 157 nm. In some cases, water or water/alcohol mixtures may be used with nonabsorbing pulsed lasers, such as excimer lasers, on silicon or metal substrates using substrate absorption. The laser heated substrate transfers energy to the energy transfer medium via conduction and effects explosive evaporation and particle removal.

However, for some substrates, such as deep ultraviolet high resolution lithography masks, the substrate is transparent at almost all of the candidate laser wavelengths, such as excimer (308 nm, 248 nm, and 193 nm), the Er:YAG (2.9404 microns), and at all visible wavelengths. As particles that produce device defects necessarily lie in the clear areas of the mask, the lasers that are available to effect substrate absorption are limited. There is one laser, the CO₂ laser at approximately 9-11 microns, which is strongly absorbed into the currently proposed fused silica substrates. These substrates for reticles for approximately 157 nm lithography, however, are modified by the addition of F doping to enhance the transmission at approximately 157 nm and are relatively thin. Substrate absorption may not allow particle removal without producing substrate damage. In such cases, absorption into the ETM using either front side or back side irradiation through the transparent substrate may provide optimized LAPR conditions.

According to the methods and apparatus of the invention, the wavelength of the laser energy, the pulse length and shape of the laser energy, the laser energy density, the laser beam size and/or shape, the laser irradiation geometry, the ambient conditions, the amount and disposition of the energy transfer medium and/or the composition of the energy transfer medium are precisely and selectively controlled. The exact parameters may be calculated for the specific application and environment, including consideration of the optical constraints of the materials and the size of the particles. The wavelength of the laser should be chosen to target either the particle, substrate, the ETM of some combination thereof. The energy density should be above the removal threshold but below the damage threshold. Further, the energy density should be sufficient to be absorbed by the particle, the substrate, or the energy transfer medium, either directly or by conduction from the sample or substrate, or some combination thereof. The pulse length of the laser is preferably sufficiently short in order to achieve the desired temperature distribution of the energy transfer medium, but not any shorter in order to decrease the likelihood of substrate damage. The laser beam shape and/or size is preferably as large as possible to clean as large an area as possible. Ideally, the laser beam is a uniformly intense beam. The irradiation geometry is chosen to optimize the energy transfer to the ETM and minimize substrate or device damage. The energy transfer medium is preferably capable of providing sufficient kinetic energy to the particle in order to remove the particle during the explosive evaporation of the energy transfer medium.

The energy transfer medium may be introduced as a uniform layer of a particular thickness onto the substrate, may be introduced so as to be condensed only in the capillary spaces under the particle, or any combination thereof, the exact selection being dependent on the substrate/particle system being used. Additionally, the composition of the energy transfer medium may be selected such that it will couple more efficiently to the laser being used.

Strongly absorbing, condensable materials may be added to the energy transfer medium to allow absorption of the laser energy into the particle/substrate/energy transfer medium system.

The energy transfer medium may be, for example, an azeotrope, which is a constant boiling mixture, wherein the composition of the mixture does not change during evaporation. However, it is not necessary to use an azeotrope mixture to control the absorption of the energy transfer medium as separately controlled dual or multiple dosers, or a constant composition non-azeotropic single doser, can be utilized to achieve the same result.

The optimum absorption geometry for the most efficient laser assisted particle removal will consist of a combination of substrate, particle, and energy transfer medium absorption as a function of the particular particle/substrate system.

Controlling the absorption of the energy transfer medium also allows irradiation from the back side of the substrate, a geometry of particular interest for masks and reticles. The laser energy can be directed through the substrate to an absorbing particle or energy transfer medium absorbed under and around the particle.

Using a near UV (excimer) wavelength to effect LAPR on fused SiO₂ substrates, an ETM consisting of an azeotrope of an absorbing molecule and water or other liquid or solvent could be utilized. One example of an azeotrope involves a constant boiling mixture consisting of approximately 9% benzyl alcohol and approximately 91% water that boils at approximately 99.9° C. Benzyl alcohol absorbs strongly at approximately 248 and 193 nm. Again, since this mixture is azeotropic, it is convenient because the composition of the mixture does not change as it is evaporated. In contrast the 90% water, 10% IPA (isopropyl alcohol) mixtures that are frequently used in excimer LAPR from Si surfaces are not azeotropic mixtures and concentration in the reservoir must be constantly monitored.

FIG. 10 illustrates a method according to the invention in the form of a flow chart. In step S1, an optical radiation source or sources and the irradiation geometry are selected. The optical radiation source(s) may be selected in accordance with a desired energy distribution, based on the particle(s)/sample system. In step S2, the composition and/or amount and disposition of an energy transfer medium is tailored to the optical radiation source(s). In step S3, the appropriate gaseous or vacuum ambient is determined for the particle(s)/sample system. In step S4, a tailored optical pulse of the optical radiation source is determined in view of the composition and/or amount of the energy transfer medium. Next, in step S5, the energy transfer medium is arranged on a surface of a sample. Finally, in step S6, either the energy transfer medium and/or the sample is irradiated with the tailored optical pulse. The energy distribution of the incident laser energy is converted by the energy transfer medium from potential to kinetic energy, and is transferred to any contaminant particles on the sample, driving them from the surface to which they have been adhered.

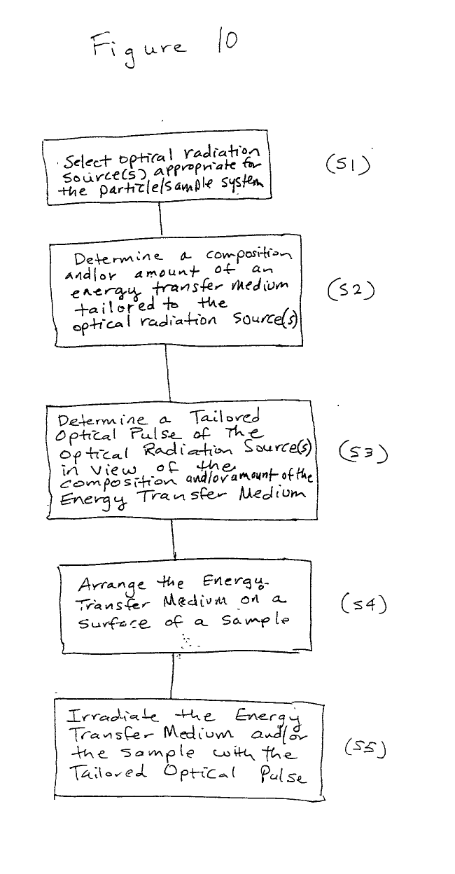

Turning now to FIG. 8, there is shown an apparatus configured to practice the present invention. The apparatus includes a sealable chamber 50 which is coupled to a vacuum source 51 for evacuating the chamber 50. Mounted on a support (not shown) in the chamber 50 is a substrate 54 to be cleaned. The substrate 54 has a surface 55 which contains contaminant particles (not shown in the scale of FIG. 8) to be removed.

For the purpose of controlling the adsorption and the deposition of liquid materials such as water, a cooling source 56 is coupled by conduit 57 to the substrate 54. As noted above, the temperature of the substrate 54 may be reduced to enhance water absorption to the surface 55.

For the purpose of dosing the surface with an energy transfer medium, using a liquid such as, for example, water or a suitably chosen mixture, a liquid source 60 is provided and is coupled by a dosing tube 61 to the surface 55 of the substrate 54. Vapor supplied by source 60, is coupled through the dosing tube 61 and applied to the surface 55 at the appropriate temperature to ensure adsorption on the surface and in the interstices under and around the contaminant particles. After water dosing, the temperature of the substrate 54 can be maintained by the cooling source 56, such that surface water desorbs while maintaining water in the interstices under and around the contaminant particles.

A laser source 64 is provided with means 66 for steering the laser beam, if necessary. A pulse tailoring unit 90 is provided in communication with the laser source 64. Input means (not shown) may be provided to allow a user to input the desired parameters to select a desired energy profile, or the parameters of the energy transfer medium. In the former case, the user would input parameters that allow the pulse tailoring unit to control the output of the laser source 64 to yield a tailored pulse having a desired energy distribution based on the application and/or environment. Alternately, the user could input the parameters of the desired optical pulse. In the latter case, the user could input the parameters of the energy transfer medium dosing pulse, and the pulse tailoring unit 90 would tailor an optical pulse to produce an energy distribution suitable for the input energy transfer medium parameters.

After a sample is prepared for cleaning and the desired parameters are input into the pulse tailoring unit 90, the laser source 64 is energized, and outputs pulses of energy as a beam 65 to the surface 55. As an alternative, the sample itself can be moved within the chamber 50 to direct the laser beam to the desired area of the surface 55. In any event, the beam 65 is focused on the areas of the surface 55 which are to be cleaned and the laser 64 pulsed to couple adequate energy to the surface 55.

As seen in FIG. 8, the sample 54 is mounted vertically such that particles (and any ETM) which are driven from the surface 55 can fall by means of gravity without redepositing on the surface. The vacuum source 51 is filtered in order to remove particles (and any ETM) which have been freed while maintaining the atmosphere within chamber 50 at a high vacuum and, therefore, clean. As an alternative, the samples 54 can be mounted horizontally with the surface 55 facing downwardly to get a further gravity assist for removal of particles once they are freed from the surface. Indeed, any mounting orientation compatible with the mechanism for removing the dislodged particles will be adequate. In most earthbound applications any orientation from the vertical illustrated in FIG. 8 to horizontally inverted will be acceptable in order to utilize a gravity assist in evacuating dislodged particles. When a system is utilized which introduces an external force for imparting particle velocity (such as the gas jet to be described below), other orientations for the surface to be cleaned might also be utilized. Alternatively, a cold plate (not shown) can be provided that draws the removed particles (and any ETM) away from the sample and prevents them from redepositing.

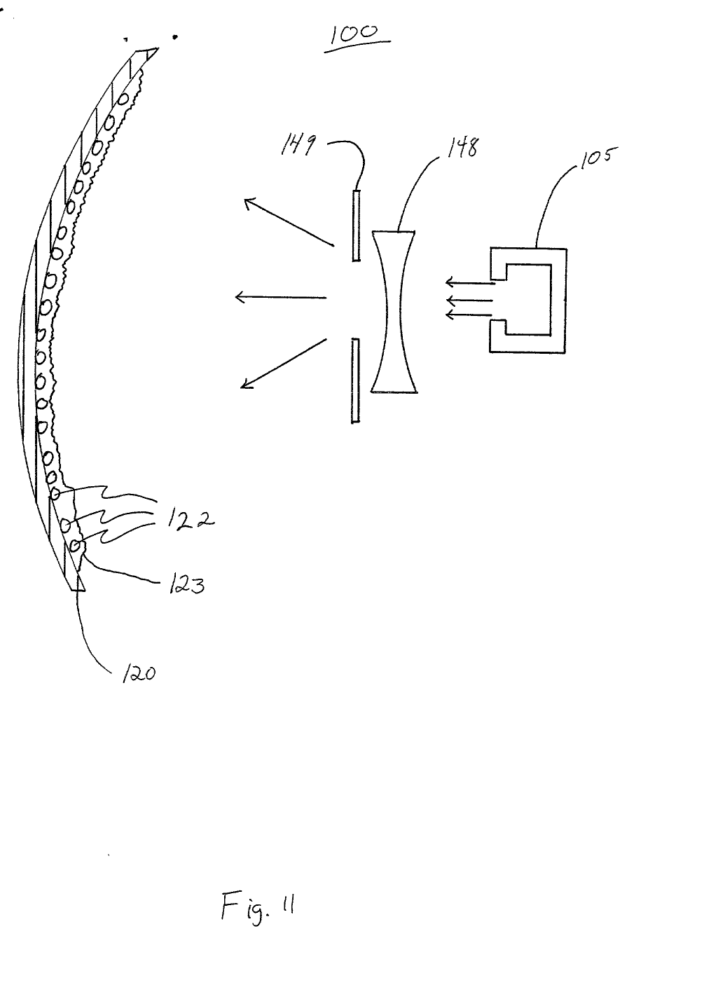

Turning now to FIG. 9, there is shown an alternative configuration adapted for removal of dislodged particles before such dislodged particles can redeposit on the surface. FIG. 9 does not contain all of the detail of FIG. 8 but instead shows only the substrate 54 having a contaminated surface 55 which is to be cleaned. The laser 65 is shown as being incident on the surface 55 which, as will be appreciated, has been dosed to provide an energy transfer medium interposed under and around the particles to be removed. Operating in conjunction with the laser 65 which dislodges the particles is a gas source 70 and an outlet conduit 71 adapted to impinge a gas jet on the surface. A vacuum source 72 having a conduit 73 directed at the surface being cleaned can also be used for drawing away particles freed by the laser 65. The system of FIG. 9 demonstrates that the invention can be practiced without a vacuum, but in most situations it will be useful to have an auxiliary mechanism, such as the gas jet, to impart a velocity to the dislodged particles to remove them from the area of the surface to avoid recontamination. This concept was disclosed in U.S. Pat. No. 5,024,968 issued to Audrey C. Engelsberg on Jun. 18, 1991, which is hereby incorporated by reference. It is noted above that in space based applications, such a mechanism may not be necessary since the velocity imparted by explosive evaporation of the energy transfer medium will impart adequate velocity to the particles to carry them away from the surface being cleaned. Thus, the system of FIG. 9 is merely exemplary of additional structures which can be used for removing particles once they are freed in the practice of the present invention.

It will now be appreciated that what has been provided is a method and means for cleaning of a substrate which has particular application in the semiconductor fabrication arts and optical arts which utilize critical optical surfaces. Such arts require the removal of contaminant particles on the order of microns in diameter and can require removal of submicron size particles. In contrast to prior techniques which suffer substantial difficulty in removing such small particles, in accordance with the present invention, the surface bearing the contaminant particles is dosed with an energy transfer medium which is interposed under and around the particles to be removed. Laser energy is then used to irradiate the surface. The laser wavelength, the pulse length of the laser energy, the laser energy, the laser beam size and/or shape, the irradiation geometry, the ambient conditions, the amount and disposition of the energy transfer medium, and/or the composition of the energy transfer medium are selected and controlled, based on application (i.e., substrate and pattern, particle composition size, and shape) and environment (i.e., external ambient composition, and pressure) considerations, to precisely control the energy deposition into the particle(s)/sample/energy transfer medium system. The laser energy coupled to the surface causes explosive evaporation of the medium, thereby creating substantial amounts of very localized energy at the site of the particles, overcoming the binding forces of the particles to the surface and freeing the particles. Means are then provided for removing the particles before redisposition can occur.

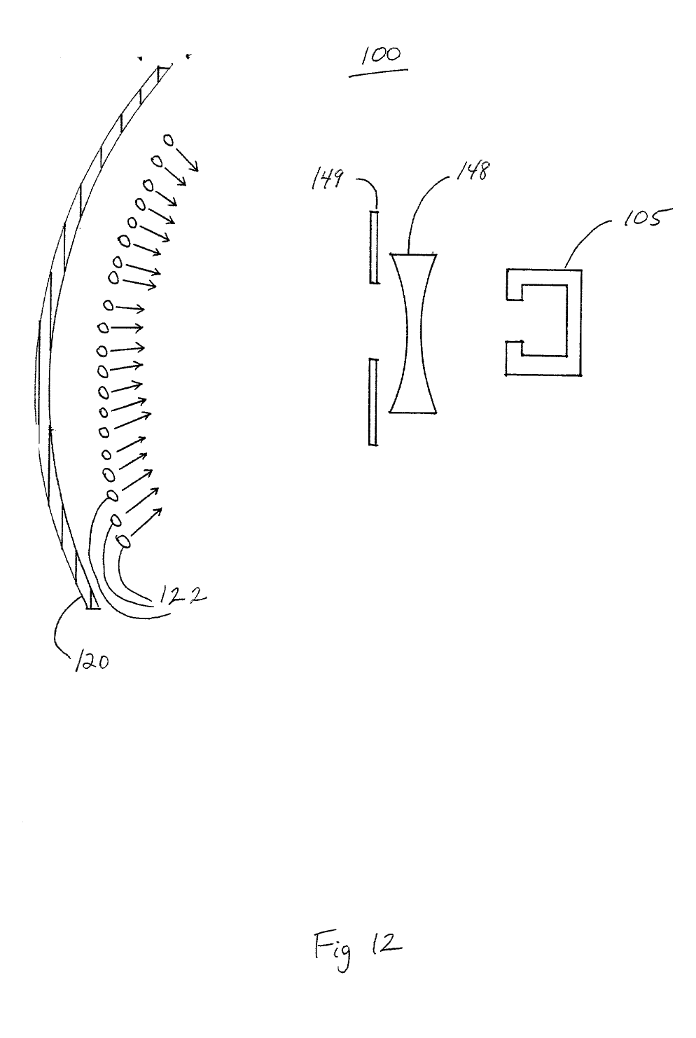

The present invention can also be used to form a particle gun, such as that shown in FIGS. 11-13, which would deposit particles onto a substrate. This can be useful in the manufacture of, for example, computer monitors. Particles interposed between a mask and a polymer, during imprinting of a polymer based diode, will create rows of pillars, creating a photonic bandgap material. See “Dusty Lab May Revolutionize LEDs”, Photonics Technology World, September 2000, which is hereby incorporated by reference. Fine control of the height and distribution of the pillars allows control of colors emitted by an LED, which are determined by microcavities in the polymer. See id. Instead of manufacturing each color with different light-emitting materials, the entire range of colors can be produced with one material by controlling the height and distribution of the pillars. See id.

The particle gun according to the invention, discussed below and shown in FIGS. 11-13, can be used to deposit particles on a substrate in a predetermined pattern and/or in layers. For example, transparent tape can be used with different kinds, sizes, etc. of particles disposed on the tape at different portions thereof. The tape can then be moved into the path of the laser energy to expose different portions of the tape to the laser energy.

The particle gun assembly 100 of FIG. 11 includes an laser energy source 105, a substrate 120, an energy transfer medium 123 (ETM), particles 122, and a target substrate 140. The laser energy source 105 can be of any wavelength and power level necessary to suitably deposit laser energy into or upon the ETM 123. The laser energy 105 can emit energy which is either coherent or incoherent, and the energy can be of a single frequency or multiple frequencies. Furthermore, the energy can be delivered to the ETM in a continuous or pulsed fashion.

The laser energy incidence angle upon the ETM can be controlled by moving the energy source 105 relative to the substrate 120 supporting the ETM 123. As shown in FIG. 11, for example, the laser energy source 105 is off-axis allowing ballistic particle deposition into a target substrate placed parallel to and some distance from the substrate 120. The laser energy can also be delivered to the ETM 123 in a time dependent manner, where different regions of the ETM 123 receive radiation at different times, such as can be effected by interposing a variable aperture 149 between the laser energy source 105 and the substrate 120 and altering the aperture 149 geometry while the substrate 120 is being illuminated or moving the substrate 120 relative to the laser. The nature of the laser energy can be further controlled by beam shaping element 148 placed between the laser energy source 105 and the substrate 120.

The substrate 120 can be formed from a wide variety of substances, including partially or completely opaque, translucent, and partially or completely transparent materials, such as metals and ceramics, plastics and resins, and glasses. The advantages of an opaque substrate include the substrate 120 acting to reflect radiation into or upon the ETM 123. The advantages of transparent substrates include allowing the laser energy source 105, and the particles 122 within the ETM 123 to be positioned on opposite sides of the substrate 120, with the radiation still reaching the ETM 123.

The substrate 120 can furthermore be formed into a wide variety of shapes such as flat, annular, spherical, hemispherical, toroidal, conic, ellipsoidal, etc. Substrate shapes can also include forms and patterns such as a linear or wire-like object formed into a circle, spiral, or a cross-hatch or screen pattern or continuous tape-like structures.

The particles 122 deposited upon the substrate 120 can include a variety of shapes, sizes, and materials. A wide range of number of particles 122 can be used with the particle gun 100, from one particle to an almost unlimited number of particles. Layers of particles of different sizes, compositions, and number densities can be produced by changing the composition of the substrate 120, the number of irradiations, the ETM, the laser energy density, and other laser energy parameters. The parameters 1)-8), previously discussed, will determine the velocity and kinetic energy of the particles. For example, a particle gun in a gaseous ambient will produce particles with velocities decreasing with increasing distances from the substrate 120 whereas ballistic particle transport is possible in vacuum ambients. Also, a uniform laser beam will produce particles with the same initial velocity.

In one embodiment of the invention, the particles 122 can further include an electrostatic charge to aid in guiding the particles 122 with an electric or magnetic field during deposition within the ETM 123, or after being accelerated by the particle gun 100. Where the particles 122 are charged, the charge can be distributed upon or within the particles 122 with various distributions to allow controlling the particle orientation during deposition upon the substrate and/or the orientation of the particles after being accelerated. Particles 122 characteristics and how they are accelerated by the particle gun 100 can further be altered by coating the particles 122 with ETM 123 before depositing them on the substrate 120 versus coating the substrate with ETM 123 before or after the particles 122 have been deposited thereon.

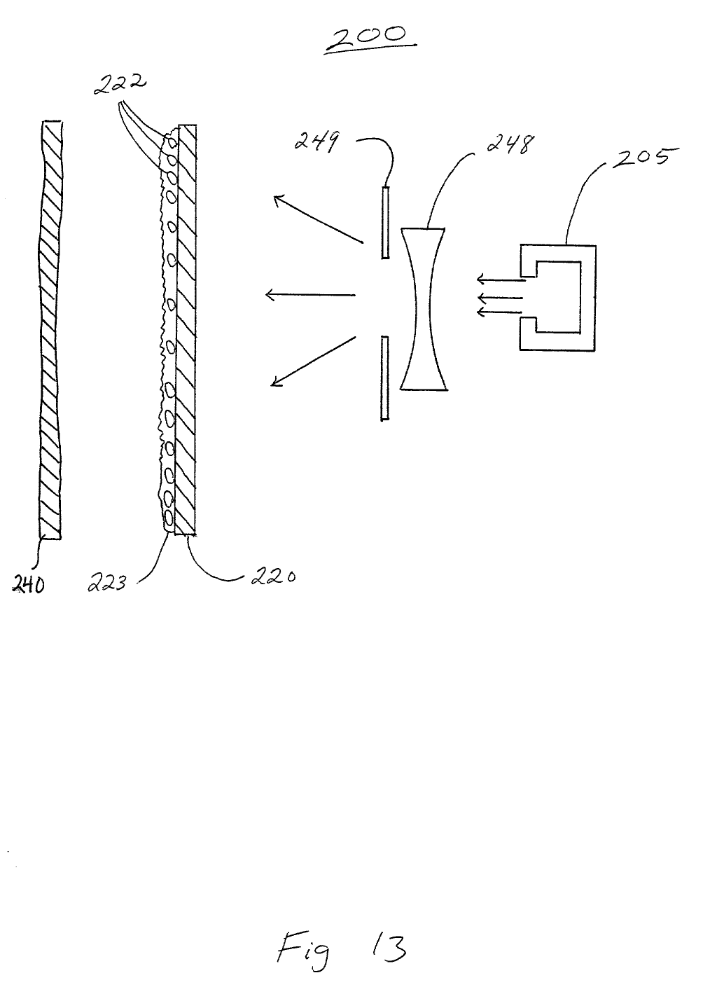

The particle gun 100 is shown in FIG. 12 after the ETM has been irradiated to launch the particles 122 away from the surface of the substrate 120 toward the target substrate 140. In this illustration, the shape of the substrate is hemispherical to achieve a focusing effect on the particles 122 to enhance the particle flux density of the particle gun 100.

The particle gun 200 of FIG. 13 includes a radiation source 205, a flat and transparent substrate 220 coated by an ETM 223. The particles 222 are distributed across the substrate 220 in a pattern where the density of particles 222 is greater towards the periphery of the substrate 220. Other particle density distributions can also be achieved. Particle density distribution can also include distributions based on particle size, mass, shape, charge, composition, etc. The particle gun 200 has its laser energy source 205 behind the substrate 220 relative to the particles 222, and also includes an aperture 249 and focusing means 248. The particle gun 200 irradiates the substrate 220, which heats the ETM 223 by conduction launching the particles 122 away from the surface of the substrate 220, and depositing the particles onto target a target substrate 240.

Patterns of particle(s) deposited by a laser particle gun can be achieved by placing the substrate 220 close to the target substrate 240 and irradiating the substrate 220 with a uniform intensity laser beam focused through a mask in photolithography, or by overlapping small focused beams. This is similar in concept to laser induced forward transfer (LIST) for generating thin film material patterns.

The foregoing embodiments and advantages are merely exemplary and are not to be construed as limiting the present invention. The present teaching can be readily applied to other types of apparatus. The description of the present invention is intended to be illustrative, and not to limit the scope of the claims. Many alternatives, modifications, and variations will be apparent to those skilled in the art. In the claims, means-plus-function clauses are intended to cover the structures described herein as performing the recited function and not only structural equivalents but also equivalent structures.

What is claimed is:

1. A method of removing one or more particle(s) adhered to a surface of a substrate, comprising:
selecting laser energy transfer parameters and a composition of an energy transfer medium based on a composition of the one or more particle(s) to be removed and a composition of the substrate;
arranging an energy transfer medium having said composition under and around the one or more particle(s) to be removed; and
irradiating at least said energy transfer medium with laser energy having said selected laser energy transfer parameters, wherein said laser energy transfer parameters and said composition of said energy transfer medium were selected to effect removal of the one or more particle(s) from the surface.

2. The method according to claim 1, wherein the step of arranging an energy transfer medium under and around the one or more particle(s) to be removed and the surface comprises adsorbing an energy transfer medium under and around the one or more particle(s) to be removed.

3. The method according to claim 1, wherein the laser energy transfer parameters comprise the wavelength of the laser energy, the density of the laser energy, the pulse length and shape of the laser energy, the pulse repetition rate of the laser energy, and the laser beam size and/or shape, and the irradiation geometry of the particle(s)/substrate/energy transfer medium.

4. The method according to claim 1, wherein the step of selecting the laser energy transfer parameters comprises selecting the wavelength of the laser energy.

5. The method according to claim 1, wherein the step of selecting the laser energy transfer parameters comprises selecting the density of the laser energy.

6. The method according to claim 1, wherein the step of selecting the laser energy transfer parameters comprises selecting the pulse length and shape of the laser energy.

7. The method according to claim 1, wherein the step of selecting the laser energy transfer parameters comprises selecting the pulse repetition rate of the laser energy.

8. The method according to claim 1, wherein the step of selecting the laser energy transfer parameters comprises selecting the laser beam size and/or shape.

9. The method according to claim 1, wherein the step of selecting the laser energy transfer parameters comprises selecting the irradiation geometry of the particle(s)/substrate/energy transfer medium.

10. The method according to claim 1, wherein the step of irradiating at least the energy transfer medium comprises irradiating the particle(s)/substrate/energy transfer medium combination.

11. The method according to claim 1, further comprising selecting ambient conditions based on a composition of the one or more particle(s) to be removed and a composition of the substrate.

12. The method according to claim 4, wherein the wavelength of the laser is selected such that the laser energy diffracts around at least some of the one or more particle(s) to be removed.

13. The method according to claim 4, wherein the wavelength of the laser is selected such that it is substantially the same size as the one or more particle(s) to be removed.

14. The method according to claim 1, wherein the composition of the energy transfer medium is selected such that it will couple efficiently with the laser energy of the laser.

15. The method according to claim 1, wherein the step of selecting the laser energy transfer parameters and the composition of the energy transfer medium comprises selecting at least one of the wavelength of the laser energy, the density of the laser energy, the pulse length and shape of the laser energy, the pulse repetition rate of the laser energy, the laser beam size and/or shape, the irradiation geometry, and/or the ambient conditions.

16. The method according to claim 15, wherein the step of selecting the laser energy transfer parameters and the composition of the energy transfer medium further comprises selecting the amount and disposition, and the composition of the energy transfer medium.

17. The method according to claim 16, wherein the laser wavelength of the laser energy, the density of the laser energy, the pulse length and shape of the laser energy, the pulse repetition rate of the laser energy, the laser beam size and/or shape, the irradiation geometry, the ambient conditions, the amount and disposition of the energy transfer medium, and/or the composition of the energy transfer medium are selected based on application and environment.

18. The method according to claim 1, wherein the laser energy is sufficient to be absorbed by the energy transfer medium, either directly or by conduction from the substrate.

19. The method according to claim 4, wherein the wavelength of the laser energy is targeted to the one or more particle(s), the substrate and/or the energy transfer medium.

20. The method according to claim 6, wherein the pulse length of the laser energy is sufficiently short in order to achieve a desired temperature distribution of the energy transfer medium.

21. The method according to claim 5, wherein the laser energy density is sufficient to be absorbed by the one or more particle(s), the substrate, or the energy transfer medium.

22. The method according to claim 8, wherein the laser beam size and/or shape is selected to clean as large a surface area as possible.

23. The method according to claim 1, wherein the energy transfer medium is a uniform layer of thickness, adsorbed under and around the one or more particle(s) to be removed, or a combination thereof.

24. The method according to claim 23, wherein the energy transfer medium is a uniform layer of thickness.

25. The method according to claim 23, wherein the energy transfer medium is adsorbed under and around the one or more particle(s) to be removed.

26. The method according to claim 1, wherein the energy transfer medium comprises a condensable material that is strongly absorbing at the selected wavelength.

27. The method according to claim 1, wherein the energy transfer material comprises an azeotrope.

28. The method according to claim 1, wherein the energy transfer material comprises separately controlled multiple dosers.

29. The method according to claim 1, wherein the energy transfer material comprises a constant composition non-azeotropic single doser.

30. The method according to claim 1, wherein the step of irradiating at least the energy transfer medium with laser energy comprises irradiating a surface of the substrate opposite to the surface containing the energy transfer medium.

31. The method according to claim 1, wherein the substrate comprises a nonabsorbing material, and the energy transfer medium comprises an absorbing mixture.

32. The method according to claim 3 1, wherein the substrate comprises at least one of SiO₂ and a CaF₂ substrate, and the energy transfer medium comprises an azeotrope of benzyl alcohol and water.

33. A method of removing one or more particle(s) adhered to a surface of a substrate, comprising:
adsorbing an energy transfer medium under and around the one or more particle(s) to be removed;
irradiating the one or more particle(s), the substrate, the energy transfer medium, or a combination thereof with laser energy; and
selecting two or more of the laser wavelength of the laser energy, the pulse length and shape of the laser energy, the density of the laser energy, the pulse repetition rate of the laser energy, the laser beam size and/or shape, the irradiation geometry, the ambient conditions, an amount and disposition of the energy transfer medium, and a composition of the energy transfer medium to precisely control an energy deposition into the one or more particle(s), the substrate, the energy transfer medium or a combination thereof; and
absorbing sufficient energy in the particle(s), the substrate, the energy transfer medium, or a combination thereof to dislodge the one or more particle(s) from the surface.

34. The method according to claim 33, wherein two or more of the laser wavelength of the laser energy, the pulse length and shape of the laser energy, the density of the laser energy, the pulse repetition rate of the laser energy, the laser beam size and/or shape, the irradiation geometry, the ambient conditions, the amount and disposition of the energy transfer medium, and the composition of the energy transfer medium are selected based on application and environment to precisely control an energy deposition into the one or more particle(s), the substrate, the energy transfer medium, or a combination thereof.

35. A method of removing one or more particle(s) from a surface of a sample, comprising:
selecting an optical radiation source having an optical energy distribution;
determining a tailored composition to serve as an energy transfer medium for said optical radiation source having said optical energy distribution; and
determining a tailored optical pulse of said optical radiation source in view of said composition, a surface of a sample, a sample and/or one or more particle(s) to be removed from a sample, such that when said energy transfer medium is arranged on the surface of the sample having the one or more particle(s) and is subsequently irradiated by said optical radiation source, sufficient energy is transferred from the tailored optical pulse to said one or more particle(s) via the energy transfer medium to dislodge said one or more particle(s) from the surface.

36. A method of removing one or more particle(s) from a surface of a sample, comprising:
determining an optical energy distribution of an optical radiation source based on the optical characteristics of a surface of a sample, a sample and/or one or more particle(s) to be removed from the sample;
tailoring a composition of an energy transfer medium in view of optical properties of said sample and said optical energy distribution;
determining a tailored pulse in view of said composition, said optical energy distribution, the surface, the sample and/or the one or more particle(s) to be removed from the sample;
applying said energy transfer medium to the surface of the sample; and
irradiating at least the energy transfer medium with the tailored pulse thereby dislodging the one or more particle(s) from the surface.

37. A method of removing one or more particle(s) from a surface of a sample, comprising:
arranging an energy transfer medium on a surface of a sample;
irradiating said energy transfer medium with a tailored optical radiation pulse, whereby energy from said tailored optical radiation pulse is absorbed largely by said energy transfer medium but not significantly by the sample causing the one or more particle(s) to be removed from the surface.

38. A system for removing one or more particle(s) from a surface of a sample, comprising:
a unit that applies an energy transfer medium on the surface of the sample; and
a tailored irradiation source that irradiates the energy transfer medium, the sample and/or the one or more particle(s) with a tailored optical radiation pulse, whereby energy from said tailored optical radiation pulse is absorbed by said energy transfer medium causing the one or more particle(s) to be removed from the surface.

39. An optical radiation source for removing one or more particle(s) from the surface of a sample having an energy transfer medium with known optical properties, comprising:
a laser source that outputs radiation having an energy distribution;
a pulse tailoring unit coupled to said laser source for forming tailored output pulses of said laser source such that, when said tailored output pulses are directed to said energy transfer medium, energy from said tailored output pulses is absorbed by the energy transfer medium causing one or more particle(s) to be removed from a surface of a sample.

40. A system for removing one or more particle(s) from a surface of a sample, comprising:
a unit that applies an energy transfer medium on the surface of the sample; and
a tailored irradiation source that irradiates the energy transfer medium, the sample, and/or the one or more particle(s), with a tailored optical radiation pulse, whereby energy from said tailored optical radiation pulse is absorbed by said energy transfer medium causing the one or more particle(s) to be removed from the surface.

41. An apparatus configured to accelerate particles, comprising:
a source of laser energy;
a substrate having a surface and a predetermined shape and configured to receive at least a portion of said laser energy from said source;
a plurality of particles arranged on said surface; and
an energy transfer medium disposed upon said surface and configured to receive at least a portion of said laser energy, whereby said plurality of particles are accelerated from said surface either by direct absorption or conduction.

42. The apparatus of claim 41, further comprising a laser energy focusing device configured to focus at least a portion of the laser energy from said source.

43. The apparatus of claim 41, further comprising a shaped aperture configured to block at least a portion of said laser energy from said source in order to create a pattern.

44. The apparatus of claim 41, further comprising means to vary an angle of incidence of the laser energy from said source upon said energy transfer medium.

45. The apparatus of claim 41, wherein said particles are distributed in a substantially uniform pattern within said energy transfer medium.

46. The apparatus of claim 41, wherein said particles are distributed in a substantially non-uniform pattern within said energy transfer medium.

47. The apparatus of claim 41, wherein said particles are substantially spherical.

48. The apparatus of claim 41, wherein said particles are randomly shaped.

49. The apparatus of claim 41, wherein said particles are hollow.

50. The apparatus of claim 41, wherein said particles comprise a metallic material.

51. The apparatus of claim 41, wherein said particles comprise a ceramic.

52. The apparatus of claim 41, wherein said particles comprise a glassy material.

53. The apparatus of claim 41, wherein said particles comprise a resinous material.

54. The apparatus of claim 41, wherein said particles comprise an electrostatic charge.

55. The apparatus of claim 41, wherein said substrate is substantially transparent.

56. The apparatus of claim 41, wherein said substrate is substantially opaque.

57. The apparatus of claim 41, wherein said substrate is substantially translucent.

58. The apparatus of claim 41, wherein said substrate comprises a metallic material.

59. The apparatus of claim 41, wherein said substrate comprises a ceramic.

60. The apparatus of claim 41, wherein said substrate comprises a glassy material.

62. The apparatus of claim 41, wherein said substrate comprises a resinous material.

63. The apparatus of claim 41, wherein said surface is planar.

64. The apparatus of claim 4 1, wherein said surface is curved.

65. A method of removing one or more particle(s) adhered to a surface of a substrate, comprising:
irradiating the one or more particle(s), the substrate, or a combination thereof with laser energy; and
selecting two or more of the laser wavelength of the laser energy, the pulse length and shape of the laser energy, the density of the laser energy, the pulse repetition rate of the laser energy, the laser beam size and/or shape, the irradiation geometry, and/or the ambient conditions to precisely control an energy deposition into the one or more particle(s), the substrate, or a combination thereof; and
absorbing sufficient energy in the particle(s), the substrate, or a combination thereof to dislodge the one or more particle(s) from the surface.

66. The method according to claim 65, wherein two or more of the laser wavelength of the laser energy, the pulse length and shape of the laser energy, the density of the laser energy, the pulse repetition rate of the laser energy, the laser beam size and/or shape, the irradiation geometry, and/or the ambient conditions are selected based on application and environment to precisely control an energy deposition into the one or more particle(s), the substrate, or a combination thereof.
